(19) United States
(12) Patent Application Publication
(10) Pub. No.: US 2007/0166836 A1
(43) Pub. Date: Jul. 19, 2007

(54) Rare earth complex excellent in thermal resistance

(76) Inventors: Kazuhiro Manseki, Osaka (JP); Yasuchika Hasegawa, Osaka (JP); Shozo Yanagida, Hyogo (JP)

Correspondence Address: HARNESS, DICKEY & PIERCE, P.L.C., P.O. BOX 8910, RESTON, VA, 20195, US

(21) Appl. No.: 10/574,778

(22) PCT Filed: Oct. 7, 2004
(86) PCT No.: PCT/JP04/14848

§ 371 Date: Feb. 27, 2007

(30) Foreign Application Priority Data
Oct. 7, 2003 (JP) 2003-348491
Mar. 8, 2004 (JP) 2004-064178

(51) Int. Cl. C07F 5/00; G01N 33/543

(52) U.S. Cl. 436/518; 534/15

(57) ABSTRACT

The present invention provides a fluorescent substance excellent in thermal resistance, which can be uniformly dispersed into plastic materials, and has a high fluorescence intensity even after experienced heat history in plastic forming processes. More specifically, the present invention provides a fluorescent substance containing a rare earth complex having benzophenone or benzoyl substituted with an alkyl group, a cycloalkyl group, an acyl group or an alkoxy group having a carbon number of 1 to 20 as a skeletal structure, in which a plurality of rare earth ions are coordinated with one or more types of molecules having a photosensitizing function.

### TECHNICAL FIELD OF THE INVENTION

The present invention relates to a rare earth complex excellent in thermal resistance, which can be uniformly dispersed into plastic materials, and has a high fluorescence intensity even after experienced heat history in plastic forming processes, a fluorescent substance containing the same, and formed resin materials containing the same.

### Background Art

Fluorescent substances are used in a variety of applications employing fluorescence emitted from them. They are compounded, for example, in paint or ink depending on purposes.

For example, JP 2002-188026A (Patent Document 1) and JP 2002-201386A (Patent Document 2) disclose fluorescent substances to be compounded in aqueous ink compositions. In these documents, an organic rare earth complex dye consisting of a rare earth element and ligands are described and thenoyltrifluoroacetone, naphthoyltrifluoroacetone, benzoyltrifluoroacetone, methylbenzoyltrifluoroacetone, and the like are listed as ligands.

Recently, it is considered to compound fluorescent substances into plastic materials in order to invest identification information. Plastic materials have been widely used as materials for food trays, industrial resin sheets and the like.

For example, fluorescent substances are compounded into plastic materials used for food trays and fluorescence emitted from these trays is detected, whereby a function identical to that of a bar code system is exerted to invest information for a source of food and the like.

In order to detect fluorescence from plastic materials in which fluorescent substances are compounded, it is required to irradiate a light with a specific wavelength, and fluorescence spectra are different depending on the fluorescent substances used. Therefore, information can be invested as a code. Accordingly, a development in code information investing technology is the center of attention.

Since plastic materials are generally formed by softening at high temperatures (e.g., about 300° C. for polycarbonate products), there are required fluorescent substances which do not decompose after heating at higher temperatures in forming and are able to emit sufficient intense fluorescence.

When inorganic fluorescent substances used in cathode ray tubes for color television such as Y₂O₃:Eu and the like are used as fluorescent substances to be compounded into plastics, although there is no problem in thermal resistance, it is a problem that inorganic fluorescent substances are impossible to uniformly disperse in plastic materials because they are insoluble in the materials. Further, fluorescence emission can be observed in an organic solvent, but it cannot be observed when compounded in plastic materials.

Therefore, it was considered to use organic rare earth complexes as disclosed in Patent Documents 1 and 2 as fluorescent substances which can be uniformly dispersed in plastic materials. However, since these fluorescent substances are not to intend for compounding into compositions requiring thermal resistance, when they are heated at temperatures needed to form plastic materials, ligands constituting complexes decompose.

Thus, in order to achieve an object to invest plastic products with fluorescence identification information, it is demanded to develop fluorescent substances excellent in thermal resistance, which can be uniformly dispersed into plastic materials, and has a high fluorescence intensity even after experienced heat history in plastic forming processes.

### DISCLOSURE OF THE INVENTION

### Problem to be Solved by the Invention

The object of the present invention is to provide fluorescent substances excellent in thermal resistance, which can be uniformly dispersed into plastic materials, and has a high fluorescence intensity even after experienced heat history in plastic forming processes.

### Means for Solving the Problem

The present inventors have made every effort to study with considering the above situation to find that a multinuclear rare earth complex, in which a plurality of rare earth ions are coordinated with additives which are conventionally compounded in plastics, surprisingly exhibit high thermal resistance that cannot be considered in conventional plastics. Consequently, the present invention has been accomplished based on the above findings.

The rare earth complex according to the present invention uses, as a ligand, plastic additives used for ultraviolet absorber. Therefore, its uniform dispersibility in plastic materials is very good, and it dose not decompose in forming plastic materials resulting in sufficient thermal resistance.

In the present invention, a “rare earth ion” means lanthanide ions, and more specifically, 14 lanthanide ions of Ce, Pr, Nd, Pm, Sm, Eu, Gd, Tb, Dy, Ho, Er, Tm, Yb and Lu. Lanthanides usually convert into a trivalent cation, but cerium (Ce) may convert into a tetravalent cation, and europium (Eu) may convert into a divalent cation.

Generally, since rare earth complexes emit fluorescence in a broad visible wavelength range and their fluorescence life times are long, they are very useful. Emission wavelengths for rare earth complexes are, for example, around 645 nm (red) for Sm, around 629 nm (red) for Eu, around 575 nm (yellowish green) for Dy, around 545 nm (green) for Tb. In addition, comparing to fluorescence life time for usual organic fluorescent compounds, of several nanoseconds, it is known that fluorescence life time for rare earth complex, especially complexes of europium (Eu) and terbium (Tb) are about several hundreds microseconds.

JP 11-256148A (Patent Document 3) discloses an illumination material in use for organic EL devises, which comprises phosphorescent substances having a triplet level and a rare earth complex, and an illumination material using a mononuclear rare earth complex in which a single europium (Eu) is coordinated with 3 molecules of dibenzoylmethane and a phosphorescence substance is illustrated. This rare earth complex receives energy from triplet excimers generated in phosphorescent substances by recombination of holes and electrons in an organic emission layer made by vacuum deposition on a substrate, and finally emits fluorescence derived from the rare earth ion.

On the other hand, the rare earth complex according to the present invention is a multinuclear complex having a plurality of rare earth ions and characterized in that it emits sufficient intense fluorescence by irradiating with ultraviolet or visible light (around 300 nm to around 450 nm) even after experienced heat history in plastic forming processes. In addition, since the multinuclear rare earth complex according to the present invention is directly excited by irradiating with ultraviolet or visible light to emit fluorescence, co-substances such as phosphorescent substances having a triplet level are not necessary.

The present invention provides:

(1) a multinuclear rare earth complex characterized in that a plurality of rare earth ions are coordinated with one or more types of molecules having a photosensitizing function to;

(2) the multinuclear rare earth complex described in (1), wherein the molecules having a photosensitizing function further have a vibrational energy quenching-suppressing function;

(3) the multinuclear rear earth complex described in (1), which is represented by the general formula:
LₚL′q(Ln)ᵣXₛ,
wherein
L is a ligand having a photosensitizing function represented by the general formula:

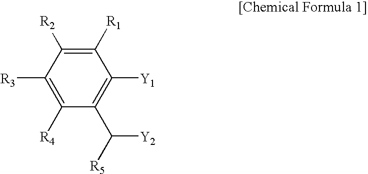

wherein R₁, R₂, R₃, R₄ and R₅ are independently hydrogen, a hydroxy group, a substituted or unsubstituted amino group, a substituted or unsubstituted aryl group, a nitro group, a cyano group, an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20;
Y₁ is —OH; and
Y₂ is ═O;
p is an integer of 1 to 40;
L′ is a ligand which is a hydroxide ion;
q is an integer of 0 to 8;
Ln is a rare earth ion;
r is an integer of 2 to 20, where a plurality of Ln may be the same or different from each other;
X is O, —OH, S, —SH, Se or Te;
s is an integer of 1 to 20, where a plurality of X may be the same or different from each other when s is an integer of 2 to 20; and further, the integers p, r and s have a relationship indicated by the expression:
1≦p/r≦4, 1≦r/s≦4 [Expression 1]
wherein a manner how L is coordinated with Ln:
Coordination Manner (A) where both Y₁ and Y₂ bind to the identical Ln; Coordination Manner (B) where Y₁ and Y₂ bind to different Ln, respectively; and a combination thereof, wherein when Ln is coordinated with Y₁, a proton leaves from —OH represented by Y₁ to form —O—, thereby Ln is coordinated with L via —O—;

(4) the multinuclear rare earth complex described in (3), wherein at least one of substituents R₁, R₂, R₃, R₄ and R₅ is an alkyl group or a cycloalkyl group represented by —R, an alkoxy group represented by —OR or an acyl group represented by —C(═O)R, where R is a substituted or unsubstituted alkyl group or cycloalkyl group having a carbon number of 1 to 20;

(5) the multinuclear rare earth complex described in (4), wherein R₅ is represented by the formula: 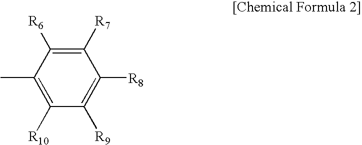
wherein R₆, R₇, R₈, R₉ and R₁₀ are independently hydrogen, a hydroxy group, a substituted or unsubstituted amino group, a substituted or unsubstituted aryl group, a nitro group, a cyano group, an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20, where at least one of R₁, R₂, R₃, R₄, R₆, R₇, R₈, R₉ and R₁₀ is an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20;

(6) the multinuclear rare earth complex described in (4), wherein R₅ is an alkyl group or a cycloalkyl group represented by —R, an alkoxy group represented by —OR, or an acyl group represented by —C(C=O)R, where R is a substituted or unsubstituted alkyl group or cycloalkyl group having a carbon number of 1 to 20;

(7) the multinuclear rare earth complex described in (5) or (6), wherein R is a substituted or unsubstituted alkyl group having a carbon number of 6 to 12;

(8) the multinuclear rare earth complex described in (7), wherein R is a substituted or unsubstituted alkyl group having a carbon number of 8 to 12;

(9) the multinuclear rare earth complex described in (1), wherein the rare earth ion is an ion of lanthanide selected from a group consisting of europium (Eu), terbium (Tb), neodymium (Nd), samarium (Sm), erbium (Er) and ytterbium (Yb) or a combination thereof;

(10) the multinuclear rare earth complex described in (5), which is represented by the general formula:
L₁₀ (Ln)₄X,
wherein
L is a ligand represented by the formula: 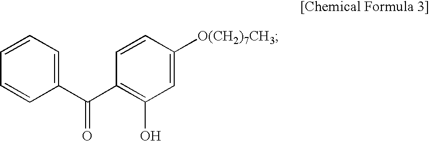
Ln is a europium (Eu) ion; and
X is O, and which has the following properties:

Elementary Analysis: as C₂₁₀H₂₅₀O₃₁Eu₄, Theoretical values C, 65.04%; H, 6.50%; Eu, 15.67% Observed values C, 64.90%; H, 6.39%; Eu, 15.41% IR (KBr, cmr⁻¹): (ν_{CH})2922, (ν_{C═C})1596, (ν_{ph-O})1243 ¹H-NMR (CDCl₃ ): δ12.7(1H,s), δ7. 6-7.2(3H,m), δ6.5-6.4(5H,d), δ4.0(2H,t), δ1.8(2H,m), δ0.9(3H,t) FAB-MS: m/z 3552.1 [Eu₄(L⁻)₉O²−]⁺;

(11) the multinuclear rare earth complex described in (5), which is represented by the general formula:
L₁₀(Ln)₄X,
wherein
L is a ligand represented by the formula: 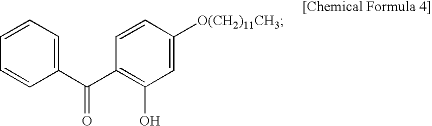
Ln is a europium (Eu) ion; and
X is O, and which has the following properties:

Elementary Analysis: as C₂₅₀H₃₃₀O₃₁Eu₄, Theoretical values C, 67.64%; H, 7.49%; Eu, 13.69% Observed values C, 67.50%; H, 7.45%; Eu, 13.49% IR (KBr, cm⁻¹): (ν_{CH})2924, (ν_{C═C})1608, (ν_{Ph-O})1247 ¹ H-NMR(CDCl₃): δ12.7(1H,s), δ7.6-7.3(3H,m), δ6.5-6.4(5H,d), δ4.0(2H,t), δ1.8(2H,m), δ0.9(3H,t) FAB-MS: m/z 4055.9 [Eu₄(L⁻)₉O²⁻]⁺;

(12) the multinuclear rare earth complex described in (6), which is represented by the general formula: L₁₆ L′₈ (Ln)₉X₂,

wherein

L is a ligand represented by the formula: 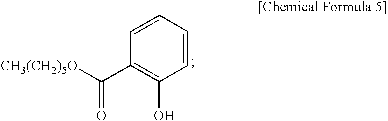
L′ is OH⁻;
Ln is a terbium (Tb) ion; and
X is O, and which has the following properties:

Elementary Analysis: as C₂₁₄H₃₂₄O₇₂NTb₉, Theoretical values C, 46.79%; H, 5.93%; Tb, 26.46% Observed values C, 46.72%; H, 5.18%; Tb, 26.04% IR (KBr, cm⁻¹): (ν_{CH})2957, 2931, (ν_{C═O})1674, 1637, (ν_{C═C})1598, (ν_{ph-O})1243 ¹H-NMR(CDCl₃): δ10.9(1H), δ7.9-6.9(4H), δ4.3(2H), δ1.8(2H), δ1.4(6H), δ0.9(3H) FAB-MS: m/z 5140.2 [Tb₉(L⁻)₁₆(O²⁻)₂(OH⁻)₈+2H+]⁺;

(13) a fluorescent substance containing the multinuclear rare earth complex described in any one of (1) to (12); and

(14) a formed resin material characterized in that the fluorescent substance described in (13) is compounded in plastic polymer.

### EFFECT OF THE INVENTION

According to the present invention, there are provided a multinuclear rare earth complex excellent in thermal resistance, which can be uniformly dispersed into plastic materials, and has a high fluorescence intensity even after experienced heat history in plastic forming processes, and a fluorescent substance containing the same. Further, according to the present invention, there is also provided formed resin materials characterized in that this fluorescent substance is compounded into plastic polymer.

The rare earth complex according to the present invention can be directly added to plastic materials, paint or ink as a fluorescent substance. A fluorescent substance prepared by mixing an organic dye (for example, coumarin and the like) with the rare earth complex according to the present invention may be added to plastic materials and the like.

A formed resin material made by using plastic materials with the fluorescent substance according to the present invention compounded therein may be used as a plastic product invested with illumination or code information. Further, since the fluorescent substance according to the present invention has a high effect on improvement of color rendering, upon compounding this fluorescent substance into a sealing resin used for LED emitting ultraviolet or visible light, a full color LED having a significantly high applicability can be produced.

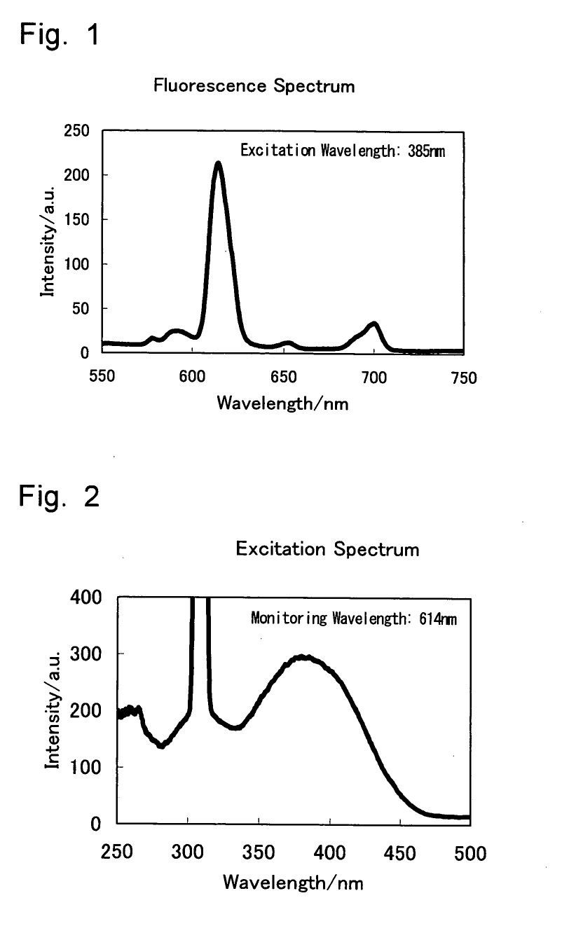

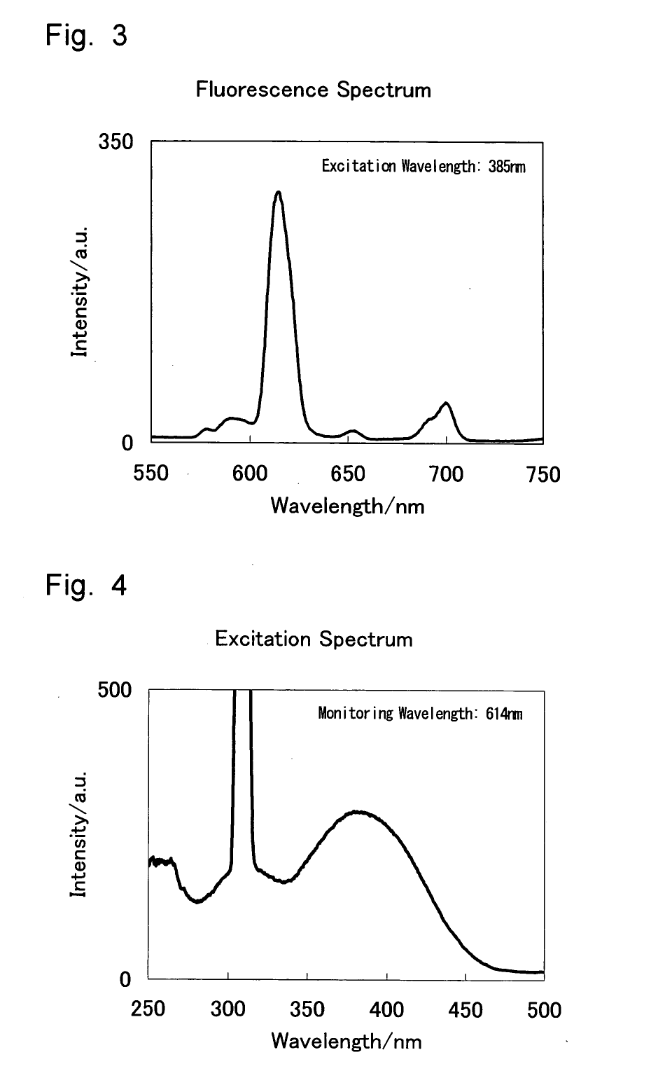

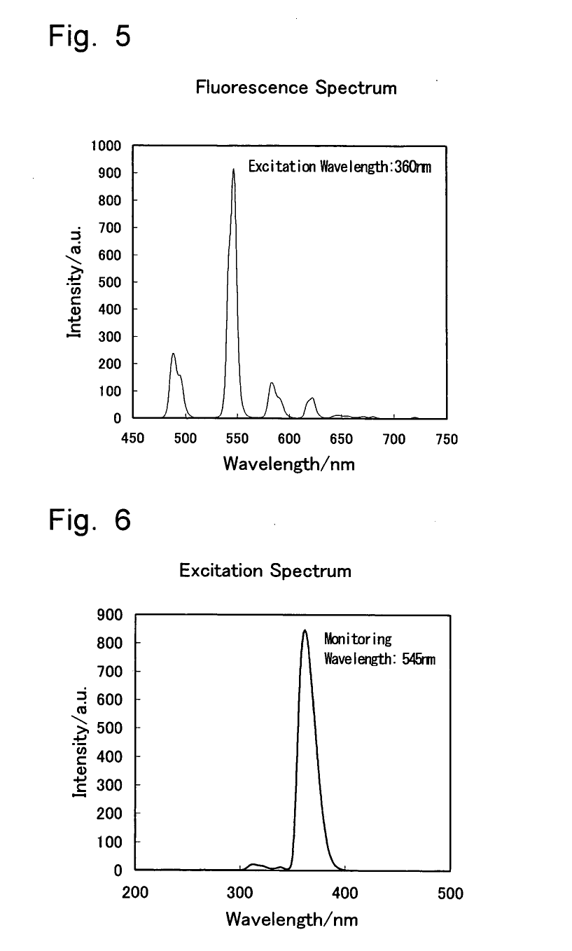

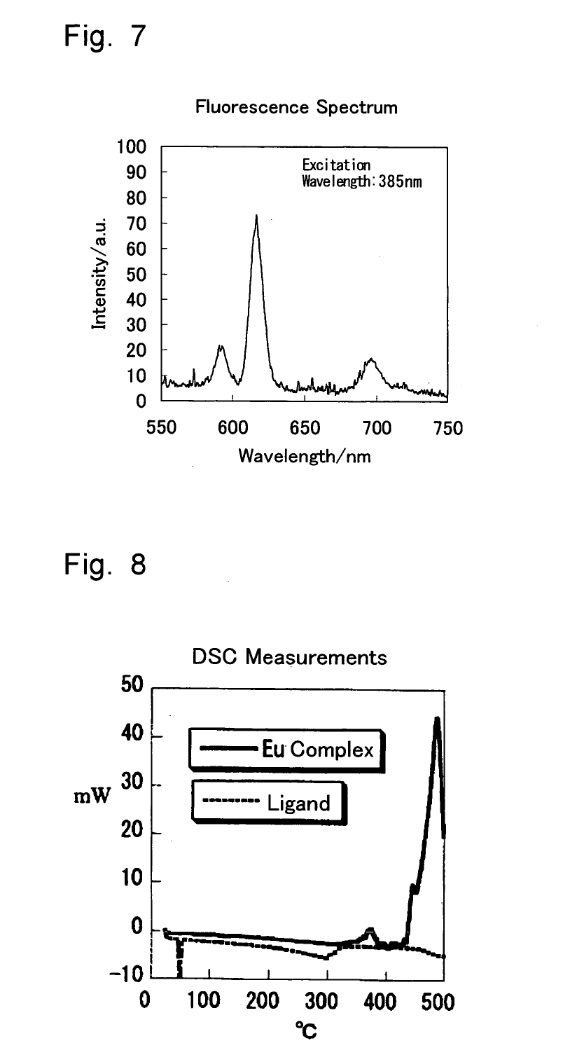

### BRIEF EXPLANATION OF THE DRAWINGS

[FIG. 1] FIG 1 shows a fluorescence spectrum for the Eu complex obtained in Example 1.

[FIG. 2] FIG. 2 shows an excitation spectrum for the Eu complex prepared in Example 1.

[FIG. 3] FIG. 3 shows a fluorescence spectrum for the Eu complex prepared in Example 2.

[FIG. 4] FIG. 4 shows an excitation spectrum for the Eu complex prepared in Example 5.

[FIG. 5] FIG. 5 shows a fluorescence spectrum for the Tb complex prepared in Example 3.

[FIG. 6] FIG. 6 shows an excitation spectrum for the Tb complex prepared in Example 3.

[FIG. 7] FIG. 7 shows an emission spectrum for the formed resin material containing the Eu complex prepared in Example 1.

[FIG. 8] FIG. 8 shows a graph which illustrates results of DSC measurements for the Eu complex prepared in Example 1 and its ligand itself.

### BEST MODE FOR CARRYING OUT THE INVENTION

The rare earth complex according to the present invention is a multinuclear rare earth complex characterized in that a plurality of rare earth ions are coordinated with one or more types of molecules having a photosensitizing function.

The rare earth ion used in the present invention is not particularly limited as far as it is a lanthanide ion, and includes, for example, europium ion Eu³⁺, terbium ion Tb³⁺, cerium ion Ce³⁺, neodymium ion Nb³⁺, samarium ion Sm³⁺, erbium ion Er³⁺, ytterbium ion Yb³⁺ and the like. A plurality of rare earth ions contained in the multinuclear rare earth complex may be the same or different from each other.

Molecules with which the rare earth ions are coordinated are those having a photosensitizing function to sensitize emission from the rare earth ions. In the present invention, a “photosensitizing function” means a function to efficiently transfer energy from irradiation to the rare earth ions.

Such molecules are, for example, a compound having benzophenone or benzoyl as a skeletal structure in which a triplet Π-Π* state exists. One or two or more types of such molecules may be contained in the complex.

The rare earth complex according to the present invention has further a vibrational energy quenching-suppressing function. In the present invention, a “vibrational energy quenching-suppressing function” means a function to suppress conversion of emission energy to thermal energy via energy transfer of the excitation state of the fluorescent substance into a vibrational structure of its surrounding media (molecules, solvents, plastics).

Examples include substituents having a long chain alkyl skeletal structure such as an alkyl group, a cycloalkyl group, an acyl group and an alkoxy group, all of which have a carbon number of 6 or greater.

Such a rare earth complex is represented by the general formula: LₚL′_{q}(Ln)ᵣXₛ. Wherein L is a ligand having a photosensitizing function, which is represented by the formula:

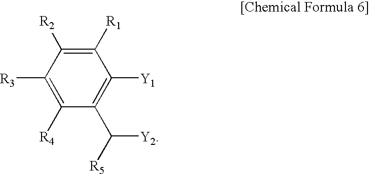

In the above formula, R₁, R₂, R₃, R₄ and R₅ are independently hydrogen, a hydroxy group, a substituted or unsubstituted amino group, a substituted or unsubstituted aryl group, a nitro group, a cyano group, an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20.

In this specification, amino groups are natural amino acids or artificial amino acids, and include, for example, glycine, alanine, leucine, tyrosine and tryptophan.

In this specification, aryl groups include, for example, a phenyl group, a tolyl group, a xylyl group, a biphenyl group, a naphthyl group, an anthryl group and a phenanthryl group.

In this specification, an alkyl group or a cycloalkyl group represented by R include, for example, a methyl group, an ethyl group, a propyl group, an isopropyl group, a hexyl group, an octyl group, a decyl group, a dodecyl group, a cyclopropyl group and a cycloheptyl group.

When the amino group is substituted, the substituents include, for example, an alkyl group, a halogen group, a nitro group, a cyano group and an aryl group.

When the aryl group is substituted, the substituents include, for example, an alkyl group, a halogen group, a nitro group, a cyano group, an alkoxy group and an acyl group.

When the alkyl group is substituted, the substituents include, for example, a halogen group, a nitro group, a cyano group, an amino group, a carboxyl group and an aryl group.

When the cycloalkyl group is substituted, the substituents include, for example, a halogen group, a nitro group, a cyano group, an amino group and an aryl group.

When the alkoxy group is substituted, the substituents include, for example, an alkyl group, a halogen group, an nitro group, a cyano group and an aryl group.

When the acyl group is substituted, the substituents include, for example, an alkyl group, a halogen group, a nitro group, a cyano group, an amino group, an alkoxy group and an aryl group,

Additionally, the alkyl group, the cycloalkyl group, the alkoxy group, the acyl group and the amino group as the above substituents are the same as defined above.

Y₁ is —OH; and Y₂ is ═O.

is an integer of 1 to 40.

L′ is a ligand different from L, and is an ordinary ligand with which a rare earth ion can be coordinated. The ligand L′ includes, for example a hydroxide ion.

q is an integer of 0 to 8, and where a plurality of L′ may be the same or different from each other when q is an integer of 2 to 8.

Ln is a rare earth ion and is not particularly limited, and includes particularly ions of lanthanides selected from a group consisting of Ce, Pr, Nd, Pm, Sm, Eu, Gd, Tb, Dy, Ho, Er, Tm, Yb and Lu, and these usually exist in a +3 valence state in a complex.

Wherein r is an integer of 2 to 20, and a plurality of Ln may be the same or different from each other.

X is an atom or an atomic group which binds to a plurality of rare earth ions to link them or binds to a single rare earth ion, and a plurality of X do not bind to each other.

X is O, —OH, S, —SH, Se or Te, s is an integer of 1 to 20, and a plurality of X may be the same or different from each other when s is an integer of 2 to 20.

Further, the integers p, r and s have a relationship indicated by the expression:
1≦p/r≦4, 1≦r/s≦4. [Expression 2]

A manner how Ln is coordinated with L to: Coordination Manner (A) where both Y₁ and Y₂ bind to the identical Ln; Coordination Manner (B) where Y, and Y₂ bind to different Ln, respectively and a combination thereof, wherein when Ln is coordinated with Y₁, a proton leaves from —OH represented by Y₁ to form —O—, thereby L coordinates to Ln via —O—.

The coordination manner between L and Ln is explained below referring to a binuclear complex in which a compound of two Ln linked via X are coordinated with one molecule of L.

When Ln is coordinated with L, a proton leaves from —OH represented by Y₁ to form —O—, thereby Ln is coordinated with L via —O—. There are Coordination Manner (A) as indicated by the structure (I): 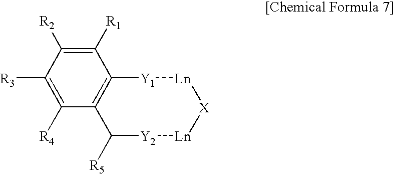
where both Y₁ and Y₂ bind to the identical Ln; Coordination Manner (B) as indicated by the structure (B): 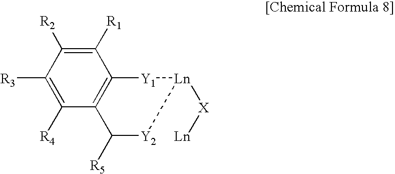
where Y₁ and Y₂ bind to different Ln each other; and a combination of Coordination Manners (A) and (B).

In addition, a plurality of Y₁ and/or Y₂ in different ligands L may bind to the identical Ln.

In the complex, the coordination binding site consisting of Y₁, Y₂ and Ln is in a resonant state.

In the ligand L in the rare earth complex according to the present invention, at least one of R₁, R₂, R₃, R₄, R₆, R₇, R₈, R₉ and R₁₀ is an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20.

In particular, R is preferably a substituted or unsubstituted long chain alkyl group having a carbon number of 6 to 20, more preferably a substituted or unsubstituted long chain alkyl group having a carbon number of 8 to 20.

In the first preferred embodiment of the rare earth complex according to the present invention, R₅ is a phenyl group represented by the formula:

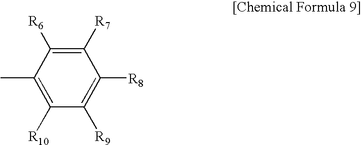

In the formula, R₆, R₇, R₈, R₉ and R₁₀ are independently hydrogen, a hydroxy group, a substituted or unsubstituted amino group, a substituted or unsubstituted aryl group, a nitro group, a cyano group, an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20.

Further, in the first preferred embodiment of the rare earth complex, at least one of R₁, R₂, R₃, R₄, R₆, R₇, R₈, R₉ and R₁₀ is an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20.

In this specification, an amino group is a natural amino acid or an artificial amino acid, and includes, for example, glycine, alanine, leucine, tyrosine and tryptophan.

In this specification, an aryl group includes, for example, a phenyl group, a tolyl group, a xylyl group, a biphenyl group, a naphthyl group, an anthryl group and a phenanthryl group.

In this specification, an alkyl group or a cycloalkyl group represented by R includes, for example, a methyl group, an ethyl group, a propyl group, an isopropyl group, a hexyl group, an octyl group, a decyl group, a dodecyl group, a cyclopropyl group and a cycloheptyl group.

When the amino group is substituted, the substituent includes, for example, an alkyl group, a halogen group, a nitro group, a cyano group and an aryl group.

When the aryl group is substituted, the substituent includes, for example, an alkyl group, a halogen group, a nitro group, a cyano group, an alkoxy group and an acyl group.

When the alkyl group is substituted, the substituent includes, for example, a halogen group, a nitro group, a cyano group, an amino group, a carboxyl group and an aryl group.

When the cycloalkyl group is substituted, the substituent includes, for example, a halogen group, a nitro group, a cyano group, an amino group and an aryl group.

When the alkoxy group is substituted, the substituent includes, for example, an alkyl group, a halogen group, an nitro group, a cyano group and an aryl group.

When the acyl group is substituted, the substituent includes, for example, an alkyl group, a halogen group, a nitro group, a cyano group, an amino group, an alkoxy group and an aryl group.

Additionally, the alkyl group, the cycloalkyl group, the alkoxy group, the acyl group and the amino group as the above substituents are the same as defined above.

In the first preferred embodiment of the rare earth complex, L is a ligand represented by the formula:

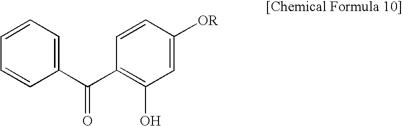

More specifically, L includes, for example, 2-hydroxy-4-octyloxybenzophenone: 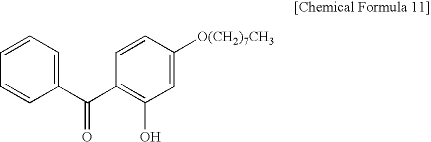
wherein R is octyl, and 4-dodecyloxy-2-hydroxybenzophenone: 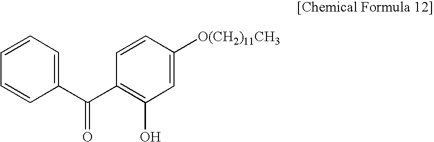
wherein R is dodecyl.

In the second preferred rare earth complex according to the present invention, L includes, for example, hexyl salicylate: 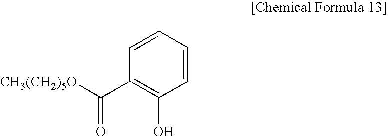
wherein R₅ is hexyloxy.

As explained above, in the preferred embodiments, the ligand has benzophenone or benzoyl as a skeletal structure.

These ligands comprise a long chain alkyl group, and due to the existence of this alkyl group, the vicinity of the rare earth complex becomes hydrophobic. When the rare earth complex is coordinated with a water molecule (a polar molecule), this quenches the excitation energy of the ligand having a photosensitizing function to reduce the emission efficiency. For example, in the case of the Eu complex coordinated with the ligands according to the first preferred embodiment, the existence of the long chain alkyl group prevents the rare earth complex from being coordinated with water molecules and, thus, these ligands are considered to have a vibrational energy quenching-suppressing function.

In addition, as will be explained below, in the Eu complex coordinated with this ligand, the excitation spectrum at 615 nm at which emission occurs corresponds to the absorption spectrum of the complex well. Accordingly, in the present complex, the ligand absorbs the photo energy and energy transfer occurs from the ligand to the rare earth ion to emit a light and, thus, the present ligand is considered to have a photosensitizing function.

The present complex may be prepared, for example, by mixing a compound which will be a ligand and a rare earth compound such as rare earth metal nitrate or rare earth metal acetate in the presence of, for example, triethylamine or lithium hydroxide.

The present complex thus prepared is insoluble in water, but very soluble in nonpolar solvent such as hexane and chloroform due to the existence of a long chain alkyl in a part of the structure. Further, it is slightly soluble in polar solvent such as methanol and acetone. Thereby, it is expected that it may be added to and well dispersed in polymer as a raw material of plastics.

The present rare earth complex may be solely added to plastic materials as a fluorescent substance. In addition, a mixture of the present rare earth complex and an organic dye for changing color (e.g., coumarin) may also be used as a fluorescent substance.

Further, the present invention also provides a formed resin material characterized in that the rare earth complex according to the present invention is compounded into plastic polymers.

The plastic polymer in which the rare earth complex is to be compounded is not limited, but includes, for example, a polyethylene resin, a polypropylene resin, a poly(vinyl chloride) resin, a urea resin, a fluorine resin, a polyester resin, a polyamide resin, a polyacetal resin, a polycarbonate resin, a polyallylate resin, a polysulfone resin, a polyphenylenesulfide resin, a polyethersulfone resin, a polyallylsulfone resin, a polytetrafluoroethylene resin, a phenol resin, an unsaturated polyester resin, an epoxy resin, a polyimide resin and a polyamideimide resin.

A method for forming is not particularly limited, but includes, for example, injection molding, blow molding, compression molding, extrusion forming, reaction forming, blow forming, heat forming, FRP forming and the like. In these methods, forming is usually carried out at 200° C. or higher and, at a higher temperature of about 300° C. for polycarbonate products.

The present complex has an excellent thermal resistance. More specifically, the Eu complex represented by the above formula exhibits thermal stability up to about 310° C. as measured by DSC (that is, the decomposition temperature is about 310° C.).

In addition, the Tb complex described above exhibits thermal stability at least up to about 200° C. Further, the Eu complex and the Tb complex are durable against water and acids, and excellent in climatic resistance due to a long alkyl chain in the ligand.

The rare earth complex according to the present invention maintains strong emission intensity after forming even when it has experienced heat history in the forming process.

### EXAMPLES

The present invention will be further specifically explained referring to the following Examples, but it should not be understood that these Examples limit the scope of the present invention.

A. Synthesis of Rare Earth Complex

### Example 1

### Synthesis of Eu Tetranuclear Complex (I)

Into methanol (100 mL), added were the ligand 2-hydroxy-4-octyloxybenzophenone (a polymer additive, seesorb 102; SHIPRO KASEI KAISHA Ltd.) represented by the formula: 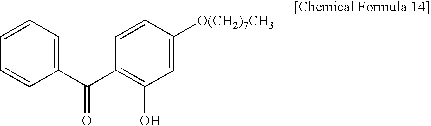
(0.6 g, 1.84 mmol) and a methanol solution of triethylamine (3.48 mL, 1.84 mmol), and after stirring for several minutes, a methanol solution (10 mL) of Eu(NO₃)₃•hexahydrate (0.32 g, 0.735 mmol) was added, and the mixture was stirred at room temperature for 2 hours. Yellow powdery crystals were obtained by suction filtration.

After washing the obtained yellow powdery crystals with methanol several times, they were analyzed by elementary analysis, IR, hu 1H-NMR and FAB-MS. Results for these analyses are shown below.

[Eu₄(L⁻)₁₀O²⁻] (Presumed Formulation)

Elementary Analysis: as C₂₁₀H₂₅₀O₃₁, Eu₄, Theoretical values C, 65.04%; H, 6.50%; Eu, 15.67% Observed values C, 64.90%; H, 6.39%; Eu, 15.41% IR (KBr, cm⁻¹): (ν_{CH})2922, (ν_{C═C})1596, (ν_{Ph-O})1243 ¹H-NMR(CDCl₃): δ12.7(1H,s), δ7.6-7.2(3H,m), δ6.5-6.4(5H,d), δ4.0(2H,t), δ1.8(2H,m), δ0.9(3H,t) FAB-MS: m/z 3552.1 [Eu₄(L⁻)₉O²⁻]⁺.

A Gd complex and a Tb complex were synthesized according to the above procedure but Gd(NO₃)₃•hexahydrate or Tb(NO₃)₃•hexahydrate was used in place of Eu(NO₃)₃•hexahydrate, and FAB-MS measurement was carried out. Molecular weights for each of rare earth elements and characteristic fragment peaks for each of the rare earth complex are shown in Table 1.

TABLE 1

| Rare Earth | Molecular | |
|---|---|---|
| Elements | Weight | Fragment Peak |
| Eu | 152.0 | 3552.1 |
| Gd | 157.3 | 3573.3 |
| Tb | 158.9 | 3580.0 |

Since properties of rare earth elements (ionic radius, coordination manner, etc.) are generally very similar to each other, it is supposed that the complexes formed have the same style. That is, it is supposed that only the center metals are replaced in those complexes.

For example, when comparing the Eu complex with the Gd complex, the differences in the fragment peaks and the molecular weights therebetween are about 21 and 5.3, respectively. Under the assumption where only the center metals are replaced in the complexes, a number of the center metals in one complex was calculated to be 4 from 21.2/5.3. Similarly, the number of the center metals was calculated to be 4 from comparisons between the Eu complex and the Tb complex, and the Gd complex and the Tb complex.

Yan et al. [C.-H Yan et al., Inorg. Chem. 41 (2002), 6802] (Non-Patent Document 1) indicated that a rare earth complex synthesized according to a process similar to that of Example 1 has a tetranuclear cross-linking structure comprising oxo linkages, which is represented by the general formula: Ln₄O, wherein Ln is a rare earth ion, based on X-ray structural analysis. From this knowledge and the above FAB-MS measurement results, the above Eu complex was suggested to be a Eu tetranuclear complex having a Eu₄O cross-linking structure.

In addition, from the results of IR measurement, there is no peak around 3400 cm⁻¹ derived from water molecules and, thereby, it is not considered that water molecules are contained in the complex as crystal water or ligands. This is consistent with the results of DSC described bellow.

Based on the above analysis results, when a ligand represented by: 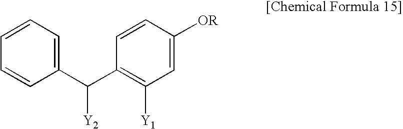
wherein R is an alkyl group having a carbon number of 8 to 12, and both Y₁ and Y₂ are O, is indicated by the following illustration: 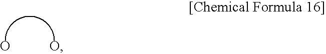
the Eu tetranuclear complex according to the present invention is presumed to be represented, for example, by

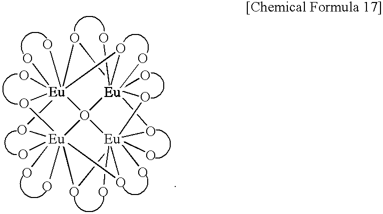

The above complex structure is merely one example to assist understanding of the structure of the present complex and, therefore, the present complex is not limited to those having this structure.

### Example 2

### Synthesis of Eu Tetranuclear Complex (II)

Into methanol (100 mL), added were the ligand 4-dodecyloxy-2-hydoxybenzophenone (a polymer additive, seesorb 102; SHIPRO KASEI KAISHA Ltd.) represented by the formula: 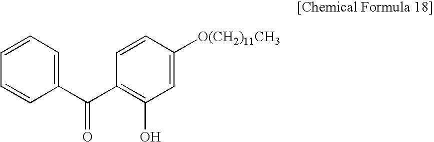
(0.6 g, 1.57 mmol) and a methanol solution of triethylamine (2.97 mL, 1.57 mmol), and after stirring for several minutes, a methanol solution (10 mL) of Eu(NO₃)₃•hexahydrate (0.280 g, 0.627 mmol) was added, and the mixture was stirred at room temperature for 2 hours. Yellow powdery crystals were obtained by suction filtration.

After washing the obtained yellow powdery crystals with methanol several times, they were analyzed by elementary analysis, IR, ¹H-NMR and FAB-MS. Results for these analyses are shown below.

[Eu₄(L⁻)₁₀O²⁻] (Presumed formulation)

Elementary Analysis: as C₂₅₀H₃₃₀O₃₁Eu₄, Theoretical values C, 67.64%; H, 7.49%; Eu, 13.69% Observed values C, 67.50%; H, 7.45%; Eu, 13.49% IR (KBr, cm⁻¹): (ν_{CH})2924, (ν_{C═C})1608, (ν_{Ph-O})1247 ¹H-NMR(CDCl₃): δ12.7(1H,s), δ7.6-7.3(3H,m), δ6.5-6.4(5H,d), δ4.0(2H,t), δ1.8(2H,m), δ0.9(3H,t) FAB-MS: m/z 4055.9 [Eu₄(L⁻)₉O²⁻]⁺.

Similar to the Eu tetranuclear complex (I) synthesized in Example 1, this complex was suggested to be a Eu tetranuclear complex having a Eu₄O cross-linking structure.

### Example 3

### Synthesis of Tb Nonanuclear Complex (III)

To a methanol solution of a ligand hexyl salicylate represented by the general formula: 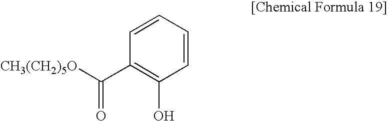
(0.600 g, 2.70 mmol), an equivalent mole amount of triethylamine (0.270 g, 2.70 mmol) was added, and after stirring for a while, a methanol solution (10 mL) of Tb(NO₃)₃•hexahydrate (0.600 g, 1.35 mmol) was added, and stirred at room temperature for 30 minutes. White crystals were obtained by suction filtration.

After washing the obtained yellow powdery crystals with methanol several times, they were analyzed by elementary analysis, IR, ¹H-NMR and FAB-MS. Results for these analyses are shown below.

[Tb₉(L⁻)₁₆(O²⁻)²(OH⁻)₈]⁻[(C₂H₅)₃NH]⁺.14H₂O (Presumed Formulation)

Elementary Analysis: as C₂₁₄H₃₂₄O₇₂NTb₉, Theoretical values C, 46.79%; H, 5.93%; Tb, 26.46% Observed values C, 46.72%; H, 5.18%; Tb, 26.04% IR (KBr, cm⁻¹): (δ_{CH})2957, 2931, (δ_{C═O})1674, 1637, (ν_{C═C})1598, (ν_{Ph-O})1243 ¹H-NMR(CDCl₃): δ10.9(1H), δ7.9-6.9(4H), δ4.3(2H), δ1.8(2H), δ1.4(6H), δ0.9(3H) FAB-MS: m/z 5140.2 [Tb₉(L⁻)₁₆(O²⁻)₂(OH⁻)₈+2H+]⁺;

A Sm complex, a Eu complex, Gd complex and a Yb complex were synthesized according to the above procedure but Sm(NO₃)₃•hexahydrate, Eu(NO₃)₃•hexahydrate, Gd(NO₃)₃•hexahydrate or Yb(NO₃)₃•hexahydrate was used in place of Tb(NO₃)₃•hexahydrate, and FAB-MS measurement was carried out. Molecular weights for each of rare earth elements and characteristic fragment peaks for each of the rare earth complex are shown in Table 2.

TABLE 2

| Rare Earth | Molecular | |
|---|---|---|
| Elements | Weight | Fragment Peak |
| Sm | 150.4 | 5063.9 |
| Eu | 152.0 | 5077.7 |
| Gd | 157.3 | 5125.8 |
| Tb | 158.9 | 5140.2 |
| Yb | 173.0 | 5267.9 |

Similar to Example 1, when comparing the Tb complex with other complexes, the difference in the fragment peaks corresponds to 9 rare earth elements and, thus, the number of the center metals was calculated to be 9.

From results of the above FAB-MS measurements, this complex was suggested to be a Tb nonanuclear complex having a polynuclear structure. With considering that Non-Patent Document 1 indicates the Ln₄O oxo cross-linking structure, it is supposed that this complex has a sandwich structure where one Tb molecule resides between two of Tb₄O cross-linking structures.

In addition, in this complex, it was presumed that quarternized triethylamime ionically bound as counter cations to form a salt.

Further, not indicated above, from results of IR measurement, a broad peak was observed around 3400 cm⁻¹derived from H₂O. Therefore, it is suggested that this complex contains water molecules. From results of DSC measurement described in Example 10, it was demonstrated that these water molecules were crystal water adsorbing to the complex in a salt form.

B. Fluorescence Properties of Rare Earth Complex in Organic Solvent

### Example 4

### Fluorescence Properties of the Eu Tetranuclear Complex (I) in Hexane

A fluorescence spectrum of the Eu complex (I) prepared in Example 1 was measured in hexane. FIG. 1 shows the fluorescence spectrum and FIG. 2 shows the excitation spectrum.

Measurements were carried out at a concentration of 1×10⁻⁴ M, slit widths of 5 nm:5 nm, and the excitation wavelength was 385 nm for the fluorescence spectrum and the monitoring wavelength was 614 nm for the excitation spectrum.

From FIG. 1 and FIG. 2, it is confirmed that the present complex exhibits fluorescence in hexane. In the fluorescence spectrum, the peak at 614 nm is derived from ⁵Do→⁷F₂ transition of Eu(III).

From the result of the excitation spectrum, when comparing with its absorption spectrum, it is considered that this peak is derived from the ligands and, thus, this complex emits light through photosensitization. In addition, it is found that the peak shown around 385 nm is derived from a Π-Π* transition of the ligands, which is observed as a result of complex formation, and the complex emits light better when excited at this wavelength.

### Example 5

### Fluorescence Properties of Eu Tetranuclear Complex (II) in Hexane

Next, a fluorescence spectrum of the Eu complex (II) prepared in Example 2 was measured in hexane. FIG. 3 shows the fluorescence spectrum and FIG. 4 shows the excitation spectrum.

Measurements were carried out at a concentration of 1×10⁻⁴ M, slit widths 5 nm:5 nm, and the excitation wavelength was 385 nm for the fluorescence spectrum and the monitoring wavelength was 614 nm for the excitation spectrum.

### Example 6

### Fluorescence Properties of the Tb Nonanuclear Complex (III) in Methanol

Next, a fluorescence spectrum of the Tb complex (III) prepared in Example 3 was measured in hexane. FIG. 5 shows the fluorescence spectrum and FIG. 6 shows the excitation spectrum.

Measurements were carried out at a concentration of 1×10⁻⁴ M, slit widths 2.5 nm:2.5 nm, and the excitation wavelength was 360 nm for the fluorescence spectrum and the monitoring wavelength was 545 nm for the excitation spectrum.

C. Fluorescence Properties of Rare Earth Complex in Formed Resin Material

### Example 7

### Fluorescence Properties of the Eu Tetranuclear Complex (I) in a Formed Polypropylene Materials

The Eu complex (I) prepared in Example 1 was compounded in polypropylene at a concentration of 100 ppm, and it was injection-molded at a resin temperature of about 200° C. to form a plate (size about 3 mm×about 48 mm × about 83 mm).

Then, the emission spectrum of this plate was measured by using a PGP detector No. 4 Type B (Refraction type) with an excitation wavelength at 385 nm. Results are shown in FIG. 7. As shown in this figure, it is found that the formed resin material in which the present rare earth complex is compounded exhibits good fluorescence emission even after experienced a heat history at a high temperature.

D. Thermal Stability of Rare Earth Complex

### Example 8

### Thermal Stability of the Eu Tetranuclear Complex (I) in the Air

In order to determine thermal stability of the Eu complex (I) prepared in Example 1, DSC measurement was carried out from room temperature to 500° C. at a heating rate of 10° C./minute using an aluminum pan. Results are shown in FIG. 8. In addition, measurement data for ligand itself are also shown for comparison.

It is found that for the ligand itself, a peak derived from its melting point is observed around 50° C., while in the complex, such peak is not detected.

Based on the measurement result (the temperature at which a peak rises), the decomposition temperature of this complex (I) was found to be 310° C.

Since regardless of the ligand itself melting around 50° C., the complex did not decompose up to over 300° C., it is considered that complexation improves thermal stability.

It is revealed that the present complexes have much higher thermal stability than a trifluoroacethylacetone Eu complex, Eu(hfac)₃, which is known to have a decomposition temperature in the air of 220° C.

The complex (I) was heated at 250° C. for about 10 minutes in the air, and after cooling, it was irradiated with a UV lamp (365 nm), and emission was observed visually.

### Example 9

### Thermal Stability of the Eu Tetranuclear Complex (II) in the Air

Similar to Example 1, DSC measurement was carried out for the complex (II) prepared in Example 2. The decomposition temperature of this complex was found to be 320° C.

### Example 10

### Thermal Stability of the Tb Nonanuclear Complex (III) in the Air

Similar to Example 1, DSC measurement was carried out for the complex (III) prepared in Example 2. The decomposition temperature of this complex was found to be 200° C.

In addition, an endothermic peak was observed around 90° C. With combining the result of IR measurement, it is considered that this endothermic peak derives from desorbing of water molecules existing in-the complex.

Hasegawa et al. [Y. Hasegawa et al., J. Phys. Chem. 100 (1996) 10201] (Non-Patent Document 2) reported that when water molecules existing in a Nd complex were ligands, the endothermic peak was observed in a higher temperature region around 130 to 160° C. Since properties of rare earth ions do not greatly vary, it is considered that when water molecules are ligands, the endothermic peak for the Tb complex is also in a temperature region around 150° C. Therefore, it is considered that the endothermic peak around 90° C. in the Tb nonanuclear complex (III) derives from desorption of crystal water adsorbing to the crystals rather than leaving of water molecules as ligands.

### Example 11

### Thermal Stability in Fluorescence Properties of the Eu Complex in a Polymer thin Film

Since it is revealed from the above DSC measurements that the Eu tetranuclear complex does not decompose even after heating at 300° C. or higher in the air, effects of the heat history on the fluorescence properties were further investigated.

Specifically, a fluorescent polymer was prepared by uniformly dispersing the Eu complex (I) or the Eu complex (II) into polyphenylsilsesquioxane (PPSQ). The mixing ratio between PPSQ and the Eu complexes was 90 wt %/10 wt %.

This fluorescent polymer was smeared on a glass substrate to form thin films. In a fluorescence life time measurement, since a film thickness does not affect the measurement, the thickness may be in a range where measurement is possible. For each of these thin films, fluorescence life times were measured at 25° C. by exciting at 380 nm and monitored at 615 nm. Further, these films were heated at 150° C., 200° C. and 250° C. for 5 minutes in a furnace, and after cooling to room temperature, fluorescence life times were measured similarly. Results are shown in FIG. 3.

Additionally, since the decomposition temperature of PPSQ itself is 500° C. or higher, this does not affect this experiment.

TABLE 3
Fluorescence Life Times for Eu Complexes
after Heating PPSQ Thin Films

| | |
|---|---|
| Heating | |
| Temperature | Fluorescence Life Time (τ) |

| for Thin Film | Eu Complex (I) | Eu Complex (II) |
|---|---|---|
| 25° C. | 0.38 ms | 0.39 ms |
| 150° C. | 0.40 ms | 0.42 ms |
| 200° C. | 0.38 ms | 0.40 ms |
| 250° C. | 0.37 ms | 0.36 ms |

Based on the above results, it is revealed that the fluorescence life times for the Eu tetranuclear complexes prepared by the present invention do not change after heating at 250° C. That is, it is confirmed that these are stable as a complex at 250° C. For comparison, polymer thin films were formed using a conventional complex Eu (hfac)₃ and they were heated at 250° C. A fluorescence life time could not be measured due to blackening.

This difference is consistent with the difference in the decomposition temperature in the air (300° C. or higher for the Eu complex (I) and the Euv complex (II), 220° C. for Eu(hfac)₃).

### INDUSTRIAL APPLICABILITY

The fluorescent substances according to the present invention may be compounded into materials requiring thermal resistance, such as plastic materials to be formed at high temperatures, to invest plastic products with fluorescence identification information.

1. A multinuclear rare earth complex formed by coordinating one or more types of molecules having a photosensitizing function and a vibrational energy quenching-suppressing function to a plurality of rare earth ions, which is represented by the general formula:
LₚL′_{q}(Ln)ᵣXₛ,
wherein
L is a ligand having a photosensitizing function represented by the general formula: 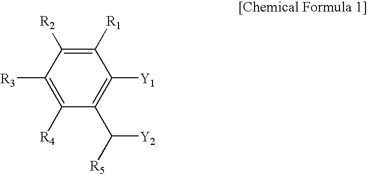
wherein R₁, R₂, R₃, R₄ and R₅ are independently hydrogen, a hydroxide group, a substituted or unsubstituted amino group, a substituted or unsubstituted aryl group, a nitro group, a cyano group, an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20;
Y₁ is —OH; and
Y₂ is ═O;
is an integer of 1 to 40;
L′ is a ligand which is a hydroxide ion;
q is an integer of 0 to 8;
Ln is a rare earth ion;
r is an integer of 2 to 20, where a plurality of Ln may be different from each other;
X is O, —OH, S, —SH, Se or Te;
s is an integer of 1 to 20, where a plurality of X may be different from each other when s is an integer of 2 to 20;
and further, the integers p, r and s have a relationship indicated by the expression:
1≦p/r≦4, 1≦r/s≦4 [expression 1]
wherein a coordination manner of L to Ln is: Coordination Manner (A) where both Y₁ and Y₂ bind to the identical Ln; Coordination Manner (B) where Y₁ and Y₂ bind to different Ln each other; and a combination thereof, wherein when Y₁ coordinates to Ln, a proton leaves from —OH represented by Y₁ to form —O—, thereby L coordinates to Ln via —O—.

2. (canceled)

3. (canceled)

4. The multinuclear rare earth complex according to claim 1, wherein at least one of substituents R1, R2, R3, R4 and R5 are an alkyl group or a cycloalkyl group represented by —R, an alkoxy group represented by —OR or an acyl group represented by —C(═O)R, where R is substituted or unsubstituted alkyl group or cycloalkyl group having a carbon number of 1 to 20.

5. The multinuclear rare earth complex according to claim 4, wherein R₅ is represented by the formula: 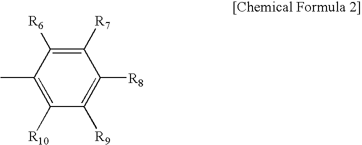
wherein R₆, R₇, R₈, R₉ and R₁₀ are independently hydrogen, a hydroxide group, a substituted or unsubstituted amino group, a substituted or unsubstituted aryl group, a nitro group, a cyano group, an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20, where at least one of R₁, R₂, R₃, R₄, R₆, R₇, R₈, R₉ and R₁₀ are an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20.

6. The multinuclear rare earth complex according to claim 4, wherein R₅ is an alkyl group or a cycloalkyl group represented by —R, an alkoxy group represented by —OR, or an acyl group represented by —C(C═O)R, where R is a substituted or unsubstituted alkyl group or cycloalkyl group having a carbon number of 1 to 20.

7. The multinuclear rare earth complex according to claim 5 or 6, wherein R is a substituted or unsubstituted alkyl group having a carbon number of 6 to 12.

8. The multinuclear rare earth complex according to claim 7, wherein R is a substituted or unsubstituted alkyl group having a carbon number of 8 to 12.

9. The multinuclear rare earth complex according to claim 1, wherein the rare earth ion is an ion of lanthanide selected from a group consisting of europium (Eu), terbium (Tb), neodymium (Nd), samarium (Sm), erbium (Er) and ytterbium (Yb) or a combination thereof.

10. The multinuclear rare earth complex according to claim 5, which is represented by the general formula:
L₁₀(Ln)₄X,
wherein
L is a ligand represented by the formula: 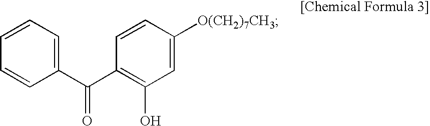
Ln is europium (Eu) ion; and
X is o, and which has the following properties:
Elementary Analysis: as C₂₁₀H₂₅₀O₃₁Eu₄, Theoretical values C, 65.04%; H, 6.50%; Eu, 15.67% Observed values C, 64.90%; H, 6.39%; Eu, 15.41% IR (KBr, cm⁻¹): (ν_{CH})2922, (ν_{c═c})1596, (ν_{ph-O})1243 ¹H-NMR(CDCl₃): δ12.7(1H,s) , δ7.6-7.2(3H,m), δ6.5-6.4 (5H,d), δ4.0(2H,t), δ1.8(2H,m), δ0.9(3H,t) FAB-MS: m/z 3552.1 [Eu₄(L⁻)₉O²⁻]⁺.

11. The multinuclear rare earth complex according to claim 5, which is represented by the general formula:
L₁₀(Ln)₄X,
wherein
L is a ligand represented by the formula: 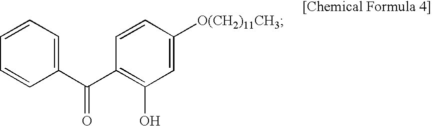
Ln is europium (Eu) ion; and
X is o, and which has the following properties:
Elementary Analysis: as C₂₅₀H₃₃₀O₃₁Eu₄, Theoretical values C, 67.64%; H, 7.49%; Eu, 13.69% Observed values C, 67.50%; H, 7.45%; Eu, 13.49% IR (KBr, cm⁻¹): (ν_{CH})2924, (ν_{C═C})1608, (ν_{Ph-O})1247 ¹H-NMR(CDCl₃): δ12.7(1H,s), δ7.6-7.3(3H,m), δ6.5-6.4(5H,d), δ4.0(2H,t), δ1.8(2H,m), δ0.9(3H,t) FAB-MS: m/z 4055.9 [Eu₄(L⁻)₉O²⁻]⁺.

12. The multinuclear rare earth complex according to claim 6, which is represented by the general formula:
L₁₆L′₈(Ln)₉X₂,
wherein
L is a ligand represented by the formula: 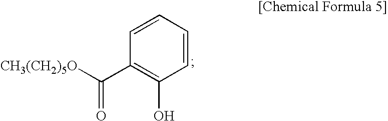
L′ is OH⁻;
Ln is terbium (Tb) ion; and
X is o, and which has the following properties:
Elementary Analysis: as C₂₁₄H₃₂₄O₇₂NTb₉, Theoretical values C, 46.79%; H, 5.93%; Tb, 26.46% Observed values C, 46.72%; H, 5.18%; Tb, 26.04% IR (KBr, cm⁻¹): (ν_{CH})2957, 2931, (ν_{C═O})1674, 1637, (ν_{C═C})1598, (ν_{Ph-O})1243 ¹H-NMR(CDCl₃): δ10.9(1H), δ7.9-6.9(4H), δ4.3(2H), δ1.8(2H), δ1.4(6H), δ0.9 (3H) FAB-MS: m/z 5140.2 [Tb₉(L⁻)₁₆(O²⁻)₂(OH⁻)₈+2H⁺]⁺.

13. A fluorescent substance containing the multinuclear rare earth complex according to any one of claims 1, and 4 to 12.

14. A resin formed materials made by compounding the fluorescent substance according to claim 13.
